(19) United States
(12) Patent Application Publication
(10) Pub. No.: US 2010/0284856 A1
(43) Pub. Date: Nov. 11, 2010

(54) GAMMA RAY RESISTANT POLYMERIC COMPOSITION

(76) Inventor: Kari Backes, Westford, MA (US)

Correspondence Address: MILLIPORE CORPORATION, 290 CONCORD ROAD, BILLERICA, MA, 01821, US

(21) Appl. No.: 12/451,806

(22) PCT Filed: Jun. 6, 2008
(86) PCT No.: PCT/US2008/007159

§ 371 Date: Jun. 23, 2010

Related U.S. Application Data
(60) Provisional application No. 60/934,261, filed on Jun. 12, 2007.

(51) Int. Cl. C08L 27/16; A61L 2/08

(52) U.S. Cl. 422/22; 525/199

(57) ABSTRACT

A polymeric composition resistant to degradation by exposure to gamma radiation comprising PVDF and between 1 and 20% by weight HDPE based on the total weight of the polymeric composition.

### CROSS-REFERENCED TO RELATED PATENT APPLICATIONS

This application claims priority to U.S. Provisional Patent Application No. 60/934,261, filed on Jun. 12, 2008 the entire contents of which are incorporated by reference herein.

### FIELD OF THE INVENTION

This invention relates to gamma ray resistant polymeric compositions. More particularly it relates to PVDF-based gamma ray resistant polymeric compositions.

### BACKGROUND OF THE INVENTION

At the present time, polymeric housings such as a filter housing or support substrate such as filter supports are commonly formed of PVDF (homopolymer of vinylidene fluoride) such as by injection molding. In order to reduce the bioburden levels in polymeric housings containing these PVDF parts after manufacture, it is commonly required that they be sterilized such as with sodium hydroxide or by exposing them to gamma radiation. When using sodium hydroxide as a sterilizing agent, it is necessary to assure it has been completely removed from the device by flushing with water prior to reusing the device. This requirement can cause difficulty such as when one or more of the parts has small crevices. In contrast, gamma radiation does not have this problem.

When utilizing PVDF, it has been found that gamma radiation causes the PVDF to degrade as evidenced by a yellowing of the PVDF. This degradation can cause the formation of elutable by-products which in many instances, such as in filtration, is undesirable. The associated color change is also undesirable in and of itself.

Accordingly, it would be desirable to provide a moldable PVDF composition which does not require sterilization with sodium hydroxide and which does not (discolor) degrade upon exposure to gamma radiation. Such a PVDF composition could be sterilized with gamma radiation while avoiding the formation of elutable by-products.

### SUMMARY OF THE INVENTION

In accordance with this invention, it has been found that a blend or alloy or compound of PVDF and HDPE (high density polyethylene) does not degrade when exposed to gamma radiation. In addition, the blend or alloy of PVDF and HDPE can be injection molded.

The blend or alloy or compound of PVDF and HDPE is formed by mixing particles of PVDF and particles of HDPE and heating the resultant homogeneous mixture to a temperature above the melting temperatures of PVDF and HDPE. The resultant melt blend then can be formed such as by extrusion or injection molding.

Likewise a process of making and gamma sterilizing a PVDF-containing product can be accomplished by the present invention by the steps of forming a polymeric composition which is resistant to degradation caused by exposure to gamma radiation which comprises a mixture of polyvinylidene fluoride (PVDF) and between about 1 and about 20% by weight high density polyethylene (HDPE) based on the total weight of said polymeric composition; melting the composition and forming it into one or more parts; assembling the parts into a final product and subjecting the final product to gamma radiation of an intensity sufficient to sterilize the final product.

### DESCRIPTION OF SPECIFIC EMBODIMENTS

The PVDF based polymeric composition comprises PVDF and from about 1 to 20% by weight, preferably between about 1% and about 5% by weight HDPE based on the total weight of the polymeric composition. Surprisingly, it has been found that such PVDF compositions are resistant to degradation caused by exposure to sterilizing gamma radiation. Sterilizing gamma radiation is generally between about 25 and about 45 kilograys of exposure.

The HDPE utilized in the present invention preferably has a molecular weight between about 100,000 and about 500,000.

The blend of PVDF and HDPE is produced by mixing particles thereof to form a homogeneous mixture. The homogeneous mixture then is heated to a temperature above the melting point of both the PVDF and HDPE. PVDF generally has a melting point within the range of 380 to 550° F. HDPE generally has a melting point within the range of 300 to 500° F. After the desired melt blend is produced, it can be formed such as by extrusion or injection molding or the like to form a part having a desired design such as a membrane substrate, a filtration cartridge housing, fitting, cap or the like.

The composition of PVDF and HDPE of this invention can be formed by grinding the individual resins into a fine powder and mixing these powders together in the appropriate ratio, and then melt processing the powder into pellets for use in injection molding. This composition would be considered essentially an alloy, in that the two resins are expected to stay homogenous on a macro level without actually becoming a compatible resin. This is comparable to microphase separation in block copolymers having blocks of incompatible polymers.

The polymeric composition of this invention optionally also can contain non elutable conventional additives such as fillers, colored pigment, blowing or foaming agent, other processing additives or the like.

The following example illustrates the present invention and is not intended to limit the same:

### Example 1

It is well known that high density polyethylene (HDPE) is gamma stable without color change. Molded product made from HDPE was gamma stable, with no color change when exposed to gamma radiation. Unpigmented PVDF when exposed to gamma radiation underwent a color change to yellow from white.

In contrast, a filtration cartridge end cap formed by molding a mixture of unpigmented PVDF and unpigmented HDPE did not undergo a change in color after being exposed to gamma radiation.

What is claimed:

1. A polymeric composition which is resistant to degradation caused by exposure to gamma radiation which comprises a mixture of polyvinylidene fluoride (PVDF) and between about 1 and about 20% by weight high density polyethylene (HDPE) based on the total weight of said polymeric composition.

2. The polymeric composition of claim 1 which contains between about 1 and 5% by weight HDPE based on the total weight of said polymeric composition.

3. An article of manufacture formed by pressure forming a melt blend of the composition of claim 1.

4. An article of manufacture formed by pressure forming a melt blend of the composition of claim 1 wherein the melt blend contains between about 1 and 5% by weight HDPE based on the total weight of said polymeric composition.

5. A process of gamma sterilizing a PVDF-based device comprising the steps of forming a polymeric composition which is resistant to degradation caused by exposure to gamma radiation which comprises a mixture of polyvinylidene fluoride (PVDF) and between about 1 and about 20% by weight high density polyethylene (HDPE) based on the total weight of said polymeric composition; melting the composition and forming it into one or more parts; assembling the parts into a final product and subjecting the final product to gamma radiation of an intensity sufficient to sterilize the final product.

6. The process of claim 5 wherein the gamma radiation is between about 25 and about 45 kilograys of exposure.

7. The composition of claim 1 wherein the mixture is a blend.

8. The composition of claim 1 wherein the mixture is an alloy.
